(19) United States
(12) Patent Application Publication
(10) Pub. No.: US 2024/0150707 A1
(43) Pub. Date: May 9, 2024

(54) NOVEL STRAIN OF LACTOBACILLUS GASSERI LM1065 SEPARATED FROM BREAST MILK, AND COMPOSITION OF RELIEVING PREMENSTRUAL SYNDROME COMPRISING THE STRAIN OR ITS CULTURE FLUID

(71) Applicant: LACTOMASON CO., LTD., Jinju-si (KR)

(72) Inventors: Minn SOHN, Jinju-si (KR); Tae Rahk KIM, Suwon-si (KR)

(21) Appl. No.: 18/412,654

(22) Filed: Jan. 15, 2024

Related U.S. Application Data
(63) Continuation of application No. PCT/KR2022/009811, filed on Jul. 7, 2022.

(30) Foreign Application Priority Data
Sep. 9, 2021 (KR) 10-2021-0120417

(51) Int. Cl. C12N 1/20; A61K 35/747; A61P 15/00; C12R 1/225

(52) U.S. Cl. CPC C12N 1/205; A61K 35/747; A61P 15/00; C12R 2001/225

(57) ABSTRACT

The present disclosure relates to a strain of Lactobacillus gasseri LM1065 (KCCM13018P) and a composition of relieving premenstrual syndrome containing the strain.

A strain of Lactobacillus gasseri LM1065 (KCCM13018P) according to an embodiment of the present disclosure can relieve premenstrual syndrome by reducing NO in blood and TL-6 gene expression and increasing the production ratio of prostaglandin E1/prostaglandin E2 in blood and DGLA levels. Therefore, the strain can be applied to food compositions, health functional food compositions, pharmaceutical compositions, and the like.

### TECHNICAL FIELD

The present disclosure relates to a strain of Lactobacillus gasseri LM1065 (KCCM13018P) separated from breast milk and a composition of relieving premenstrual syndrome containing the strain.

### BACKGROUND

Premenstrual syndrome (PMS) refers to a series of symptoms characterized by emotional, behavioral, and physical symptoms that occur repeatedly before menstruation and includes various physical symptoms such as menstrual pain and psychological changes such as mood swings, feeling depressed, anxious, or aggressive. These symptoms become progressively worse after ovulation and are most severe one week before menstruation and disappear within a few days of the onset of menstruation.

Among the typical physical symptoms, menstrual pain is a common gynecological symptom that every woman of childbearing age experiences periodically every month until she reaches menopause and is a very common symptom in the female menstrual cycle.

According to a study, 77% to 94% of Korean women complained of menstrual pain. Also, 47% of these women experienced menstrual pain every month and 53.2% experienced severe menstrual pain. Further, 46% of female manufacturing workers reported that they were restricted from their activities due to menstrual pain. In foreign countries, the prevalence of menstrual pain was as high as 60% to 93%, and 42% of the respondents experienced severe menstrual pain. Furthermore, 10% to 50% of female students reported that they were restricted from their daily activities including school activities due to menstrual pain. In the industrial fields, damage caused by menstrual pain was estimated at 600 million hours of work loss and $2 billion of productivity loss every year.

Therefore, the results of studies on the changes or mechanisms of menstrual pain-related factors can be used as sufficient evidence for the development of premenstrual syndrome-related food materials, health functional food materials and drugs.

A composition of improving premenstrual syndrome symptom containing compounds isolated from Hordeum vulgare extract (Korean Patent No. 10-2187335) is an example of a composition developed for relieving premenstrual syndrome through the change of prolactin. However, there is still a need for in-depth development and numerous studies on compositions having excellent effects of relieving premenstrual syndrome.

Accordingly, the present inventors have made efforts to develop an excellent composition capable of relieving premenstrual syndrome, and as a result, have completed the present disclosure by developing a novel strain capable of reducing NO and TL-6 gene expression and increasing the production ratio of prostaglandin E1/prostaglandin E2 in blood and DGLA levels.

### DISCLOSURE OF THE INVENTION

### Problems to be Solved by the Invention

The present disclosure is conceived to provide a novel strain of Lactobacillus gasseri LM1065 (KCCM13018P) separated from breast milk and a composition of relieving premenstrual syndrome, containing the strain.

However, the problems to be solved by this disclosure are not limited to those mentioned above, and other problems not mentioned will be clearly understood by a person with ordinary skill in the art from the following description.

### Means for Solving the Problems

A first aspect of the present disclosure provides a composition comprising a strain of Lactobacillus gasseri LM1065 (KCCM13018P).

A second aspect of the present disclosure provides a food composition of relieving menstrual pain, containing one or more of a strain of Lactobacillus gasseri LM1065 (KCCM13018P), and culture fluid, fragments and extracts of the strain as active ingredients.

A third aspect of the present disclosure provides a pharmaceutical composition of preventing or treating premenstrual syndrome, containing one or more of a strain of Lactobacillus gasseri LM1065 (KCCM13018P), and culture fluid, fragments and extracts of the strain as active ingredients.

### Effects of the Invention

A strain of Lactobacillus gasseri LM1065 (KCCM13018P) according to an embodiment of the present disclosure can relieve premenstrual syndrome by reducing NO in blood and IL-6 gene expression and increasing the production ratio of prostaglandin E1/prostaglandin E2 in blood and DGLA levels. Therefore, the strain can be applied to food compositions, health functional food compositions, pharmaceutical compositions, and the like.

### BRIEF DESCRIPTION OF THE DRAWINGS

FIG. 1 compares the NO production capacity between a strain treatment group of the present disclosure and a positive control group (PC) in a test to determine the effect of Lactobacillus gasseri LM1065 on relieving symptoms of premenstrual syndrome according to an example of the present disclosure.

FIG. 2 compares the IL-6 gene expression capacity between a strain treatment group of the present disclosure and a positive control group (PC; Positive Control) in a test to determine the effect of Lactobacillus gasseri LM1065 on relieving symptoms of premenstrual syndrome according to an example of the present disclosure.

FIG. 3 shows the result of analysis of PGE1/PGE2 ratio by ELISA on blood collected from rats from 3 days before the last administration of the test material in a test to determine the effect of Lactobacillus gasseri LM1065 on relieving symptoms of premenstrual syndrome according to an example of the present disclosure.

FIG. 4 shows the result of analysis of the concentration of DGLA by Liquid Chromatograph-Tandem Mass Spectrometry (LC-MS/MS) on serum collected on the last day of administration of the test material in a test to determine the effect of Lactobacillus gasseri LM1065 on relieving symptoms of premenstrual syndrome according to an example of the present disclosure.

### BEST MODE FOR CARRYING OUT THE INVENTION

Hereafter, examples of the present disclosure will be described in detail with reference to the accompanying drawings so that the present disclosure may be readily implemented by a person with ordinary skill in the art. However, it is to be noted that the present disclosure is not limited to the examples but can be embodied in various other ways. In the drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected to” another element and an element being “electronically connected to” another element via another element.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Hereinafter, embodiments and examples of the present disclosure will be described in detail. However, the present disclosure may not be limited to the following embodiments and examples.

A first aspect of the present disclosure provides a composition comprising a strain of Lactobacillus gasseri LM1065 (KCCM13018P).

In an embodiment of the present disclosure, the strain may reduce the production amount of NO in blood.

Through the whole document, the term “NO (nitric oxide)” refers to a compound in which nitrogen is oxidized, which is called “nitrogen oxide” or “nitrogen monoxide”. It is formed from arginine, which is an amino acid, in cells, and serves as a kind of signal transmitter and is involved in various physiological activities such as immune reaction, vasodilation and signal transmission. It is also known to induce inflammation and pain by promoting the secretion of inflammatory cytokines such as TNF-α and IL-6.

The strain of Lactobacillus gasseri LM1065 of the present disclosure can reduce the production amount of NO and thus relieve menstrual pain.

Through the whole document, the term “Griess reagent” refers to a reagent used in a Griess test to determine the presence or absence of nitrite ions NO2-. In general, it is prepared by mixing a solution of 0.5 g of sulfanilic acid in 150 ml of dilute acetic acid with a solution of 0.1 g of a-naphthylamine in 20 ml of water and 150 ml of acetic acid. The NO production capacity measurement test is a test using the Griess reagent, and is performed to measure the expression level of NO.

In an embodiment of the present disclosure, the strain may reduce IL-6 gene expression.

Through the whole document, the term “IL-6 (InterLeukin-6)” together with TNF-α (Tumor Necrosis Factor-a) refers to a physiologically active protein called a cytokine. IL-6 is secreted from various cells such as T lymphocytes and macrophages to promote immune responses. In particular, IL-6 is involve d in inflammatory responses and causes inflammation and pain in the process of regulating high fever or acute phase proteins.

The strain of Lactobacillus gasseri LM1065 of the present disclosure can reduce IL-6 gene expression and thus relieve menstrual pain.

Through the whole document, the term “LPS (lipopolysaccaharide)” refers to a representative pathogenic substance that induces macrophage-mediated inflammation. LPS binds to Toll-like receptor 4 (TLR4), which is expressed on the surfaces of macrophages, and induces inflammation through secretion of inflammatory cytokines and substances. Such inflammation is also induced by inflammasomes present in cells. The activation of inflammasomes causes an increase in pyroptosis, which is an inflammatory form of cell death, and secretion of interleukin-1β (IL-1β) and interleukin-18 (IL-18), which are inflammatory cytokines. In macrophage-mediated inflammation, mouse caspase-11 and its human homologues, caspase-4/5, are also known to induce pyroptosis and secretion of IL-1β and IL-18, which directly recognize intracellular LPS derived from Gram-negative bacteria and induce inflammation (Young Soo Lee, 2017).

Through the whole document, the term “prostaglandin” refers to a kind of hormone secreted during menstruation and serves to contract the uterus and causes menstrual blood to be smoothly pushed out when the endometrium is peeled off.

Prostaglandin is converted from arachidonic acid by a cyclooxygenase (COX) system. Prostaglandin possessing various physiological activities is produced through conversion from arachidonic acid into prostaglandin G2 and then into prostaglandin H2. Also, prostaglandin has been reported to cause inflammation and pain.

Through the whole document, the term “PGE2 (prostaglandin E2)”, also known as “dinoprostone”, is a naturally occurring prostaglandin with oxytocic properties. [37] PGE2 acts through G protein-coupled PGE2 receptors that induce inflammation-mediated pain in damaged tissues or cells and stimulate the central nervous system and the peripheral nervous system.

In an embodiment of the present disclosure, the strain may increase the ratio of prostaglandin E1/prostaglandin E2 in blood. Specifically, the strain may increase the production ratio of prostaglandin E1 to prostaglandin E2.

Through the whole document, the term “PGE1 (prostaglandin E1)”, also known as “alprostadil”, is a naturally occurring prostaglandin which serves to dilate blood vessels in the body, and is known to induce smooth muscle relaxation and attenuate the secretion of inflammatory cytokines and thus relieve pain.

The production ratio of PGE1/PGE2 (PGE1 to PGE2) is a major indicator according to the guidelines from the Ministry of Food and Drug Safety for women with premenstrual syndrome. An increase in the production ratio is considered to have a function related to treatment of premenstrual syndrome.

Accordingly, it can be seen that the strain of Lactobacillus gasseri LM1065 of the present disclosure can increase the production ratio of prostaglandin E1/prostaglandin E2 in blood and thus relieve menstrual pain and premenstrual syndrome.

In an embodiment of the present disclosure, the strain may increase the level of GLA or DGLA in blood.

Through the whole document, the term “GLA (Gamma-linolenic acid)” refers to a fatty acid found primarily in vegetable oils.

Prostaglandin metabolism disorders are found in women with premenstrual syndrome. Prostaglandins are involved in central nervous system function, fluid balance, and uterine contractility regulation, and, thus, prostaglandin metabolism disorders can cause premenstrual syndrome. Therefore, it is known that allowing women with defects in the process of converting linoleic acid to gamma-linolenic acid in the prostaglandin synthesis process to take GLA can directly synthesize prostaglandin, which can help relieve the symptoms of premenstrual syndrome.

Through the whole document, the term “DGLA (Dihomo-gamma-linolenic acid)” refers to an omega-6 fatty acid composed of 20 carbon atoms and is mainly contained in plant seed oils. When linoleic acid, an essential fatty acid, is consumed, GLA is synthesized slowly and then GLA is quickly converted to DGLA.

DGLA produces PGE 1 and 15-OH-DGLA through metabolism. As described above, PGE 1 is effective in suppressing inflammation, and 15-OH-DGL suppresses 5-lipoxygenase and 12-lipoxygenase to inhibit the production of inflammatory metabolites, such as PGE2 and LTB4, produced from arachidonic acid.

The strain of Lactobacillus gasseri LM1065 of the present disclosure may enhance the production of GLA and DGLA and thus relieve menstrual pain.

In an embodiment of the present disclosure, the strain may relieve menstrual pain. Specifically, the strain may be contained in various compositions such as a food composition, a health functional food composition and a pharmaceutical composition of relieving menstrual pain.

A second aspect of the present disclosure provides a food composition of relieving menstrual pain, containing one or more of a strain of Lactobacillus gasseri LM1065 (KCCM13018P), and culture fluid, fragments and extracts of the strain as active ingredients. The features described above in respect of the first aspect of the present disclosure may equally apply to the food composition according to the second aspect of the present disclosure.

Through the whole document, the term “relieve” refers to all activities reducing menstrual pain or improving related symptoms by administering the composition.

In an embodiment of the present disclosure, the composition may relieve menstrual pain. Specifically, the composition may relieve menstrual pain by reducing NO and prostaglandin E2 in blood and increasing the production ratio of prostaglandin E1/prostaglandin E2 in blood.

In an embodiment of the present disclosure, the composition may contain Lactobacillus gasseri LM1065, live bodies, heat-killed bodies, culture fluid, fragmented products and/or or extracts thereof.

Through the whole document, the term “heat-killed bacteria” is opposite to the term “live bacteria” and refers to bodies obtained by suppressing the growth of bacteria such as heat-treating live bacteria obtained by fermentation and metabolites thereof. The heat-killed bacteria may contain cytoplasm, cell wall, antibacterial substances such as bacteriocin, polysaccharides, organic acid, and the like. Products using the heat-killed bacteria have higher stability than live bacteria products and are particularly excellent in heat resistance and have high stability to the external environment. Therefore, the products using the heat-killed bacteria are easier to store and have longer shelf life than the existing live bacteria products. Further, since the regulations on the use of antibiotics become stricter, there are a handful of companies that have produced heat-killed bacteria products. Therefore, considering the application as substitutes and the number of the producing companies, the marketability and growth potential is very high.

Through the whole document, the term “culture fluid” refers to a substance obtained by culturing the strain of the present disclosure in a known liquid medium or solid medium and may be interchangeably used with “culture”.

In an embodiment of the present disclosure, the composition may be a food composition or a health functional food composition.

Through the whole document, the term “food” may include meats, sausages, breads, chocolates, candies, snacks, cookies, pizza, ramens, other noodles, gums, dairy products including ice cream, soups, beverages, tea, drinks, alcohol drinks, vitamin complexes, health functional food and health food, and may include all foods in the accepted meaning.

Through the whole document, the term “health functional food” refers to foods prepared and processed using raw materials or ingredients having useful functions in accordance with Korean Health Functional Foods Act, No. 6727, which means that it is ingested for the purpose of obtaining a beneficial effect for health use such as control of nutrients or physiological action for the structure and function of the human body.

The food of the present disclosure can be manufactured by conventional methods used in the art, and can be manufactured by adding conventional raw materials and ingredients used in the art. Further, a formulation of the food is not limited as long as the formulation is accepted as a food. The food composition of the present disclosure may be prepared as a variety of formulations. Since the food is used as raw materials, unlike general drugs, the food composition is free from side effects which may occur when a drug is taken for a long time, and may have excellent portability. Therefore, the food of the present disclosure may be taken as a supplement for enhancing the effects of improving the intestinal environment.

The health food refers to a food having effects of actively maintaining or promoting health conditions, as compared with general foods, and a health supplement food refers to a food for supplementing health. If necessary, the health functional food, health food, and health supplement food may be interchangeably used with each other. Specifically, the health functional food is a food prepared by adding Lactobacillus gasseri LM1065 of the present disclosure to food materials such as beverages, teas, spices, gums, confectionery, etc., or prepared as a capsule, a powder, a suspension, etc. The health functional food means that it has a specific effect on health when consumed, but unlike general drugs, the health functional food is free from side effects that may occur when a drug is taken for a long time since the food is used as raw materials.

Since the food composition of the present disclosure can be routinely ingested, the food composition is expected to show a high efficacy on the prevention or improvement of the intestinal environment and thus can be very usefully applied.

The food composition may further contain a physiologically acceptable carrier. The kind of the carrier is not particularly limited. Any carrier may be used as long as it is commonly used in the art.

Further, the food composition may further contain additional ingredients that are commonly used in food compositions so as to improve smell, taste, visuality, etc. For example, the food composition may contain vitamins A, C, D, E, B1, B2, B6, B12, niacin, biotin, folate, pantothenic acid, etc. Furthermore, the food composition may also contain minerals such as zinc (Zn), iron (Fe), calcium (Ca), chromium (Cr), magnesium (Mg), manganese (Mn), copper (Cu), etc. Moreover, the food composition may also contain amino acids such as lysine, tryptophane, cysteine, valine, etc.

Further, the food composition may also contain food additives, such as preservatives (potassium sorbate, sodium benzoate, salicylic acid, sodium dehydroacetate, etc.), disinfectants (bleaching powder, higher bleaching powder, sodium hypochlorite, etc.), antioxidants (butylhydroxyanisole (BHA), butylhydroxytoluene (BHT), etc.), colorants (tar color, etc.), color-developing agents (sodium nitrite, etc.), bleaching agents (sodium sulfite), seasonings (monosodium glutamate (MSG), etc.), sweeteners (dulcin, cyclemate, saccharin, sodium, etc.), flavors (vanillin, lactones, etc.), swelling agents (alum, potassium D-bitartrate, etc.), fortifiers, emulsifiers, thickeners (adhesive pastes), film-forming agents, gum base agents, antifoaming agents, solvents, improvers, etc. The additives may be selected and used in an appropriate amount depending on the type of food.

Lactobacillus gasseri LM1065 may be added as it is, or may be used in conjunction with other foods or food ingredients according to a conventional method. The mixing amount of active ingredients may be appropriately determined depending on the purpose of use (prophylactic, health or therapeutic treatment). In general, when a food or a beverage is manufactured, the food composition of the present disclosure may be added in an amount of 50 parts by weight or less, specifically 20 parts by weight or less based on the total weight of the food or the beverage. However, when taken for the purpose of health and hygiene, the food composition may be contained in an amount below the range. In addition, since there is no safety problem, the active ingredients may be used in an amount above the range.

The food composition of the present disclosure may be used as, for example, a health beverage composition, and in this case, the health beverage composition may further contain various flavors or natural carbohydrates, as in common beverages. The natural carbohydrates may include monosaccharides such as glucose and fructose; disaccharides such as maltose and sucrose; polysaccharides such as dextrin and cyclodextrin; and sugar alcohols such as xylitol, sorbitol and erythritol. The sweeteners may be natural sweeteners such as thaumatin or a stevia extract; or synthetic sweeteners such as saccharin or aspartame. The natural carbohydrate may be generally used in an amount of from about 0.01 g to about 0.04 g, and specifically, from about 0.02 g to about 0.03 g based on 100 mL of the health beverage composition of the present disclosure.

In addition, the health beverage composition may contain various nutrients, vitamins, minerals, flavors, colorants, pectic acid and salts thereof, alginic acid and salts thereof, organic acid, protective colloidal thickeners, pH regulators, stabilizers, antiseptics, glycerin, alcohols, carbonating agents, etc. Moreover, the health beverage composition may contain fruit flesh used to prepare natural fruit juices, fruit juice beverages, or vegetable beverages. These ingredients may be used individually or in combination. A proportion of the additives is not critical, but is generally selected from 0.01 part by weight to 0.1 part by weight per 100 parts by weight of the health beverage composition of the present disclosure.

The food composition of the present disclosure may contain Lactobacillus gasseri LM1065 of the present disclosure in a variety of % by weight as long as it can exhibit the effect of improving the intestinal environment. Specifically, Lactobacillus gasseri LM1065 of the present disclosure may be contained in an amount of 0.00001% by weight to 100% by weight or 0.01% by weight to 80% by weight based on the total weight of the food composition but may not be limited thereto.

In an embodiment of the present disclosure, the food composition may be a health functional food composition.

A third aspect of the present disclosure provides a pharmaceutical composition of preventing or treating premenstrual syndrome, containing one or more of a strain of Lactobacillus gasseri LM1065 (KCCM13018P), and culture fluid, fragments and extracts of the strain as active ingredients. The features described above in respect of the first and second aspects of the present disclosure may equally apply to the pharmaceutical composition according to the third aspect of the present disclosure.

In an embodiment of the present disclosure, the composition may contain a strain of Lactobacillus gasseri LM1065, live bodies, heat-killed bodies, culture fluid, fragments and/or extracts of the strain.

Through the whole document, the term “treat” refers to all activities improving or enhancing premenstrual syndrome by administering a pharmaceutical composition containing Lactobacillus gasseri LM1065 of the present disclosure as an active ingredient to a subject with premenstrual syndrome.

In an embodiment of the present disclosure, the composition may function to treat or prevent premenstrual syndrome. Specifically, it may treat or prevent premenstrual syndrome by reducing prostaglandin E2 in blood and increasing the production ratio of prostaglandin E1/prostaglandin E2.

Through the whole document, the term “premenstrual syndrome” refers to a series of symptoms characterized by emotional, behavioral and physical symptoms that occur repeatedly before menstruation and includes edema, breast pain, digestive disorders, headache, back pain, lower abdominal pain, abdominal distention, constipation, diarrhea, depression and insomnia.

In an embodiment of the present disclosure, the pharmaceutical composition may be formulated and used as formulations for oral administration such as powders, granules, tablets, capsules, suspensions, emulsions, syrups or aerosol, external preparations, suppositories or sterile injection solutions by conventional methods, respectively, but may not be limited thereto.

In an embodiment of the present disclosure, the pharmaceutical composition may be formulated with generally used diluents or excipients such as fillers, extenders, binders, wetting agents, disintegrating agents or surfactants, but may not be limited thereto.

In an embodiment of the present disclosure, solid formulations for oral administration may include tablets, pills, powders, granules or capsules, and these solid formulations may be prepared by mixing heat-killed bodies of Lactobacillus gasseri with at least one of excipients such as starch, calcium carbonate, sucrose, lactose or gelatin. Except for the simple excipients, lubricants such as magnesium stearate or talc may be used, but the present disclosure may not be limited thereto.

In an embodiment of the present disclosure, liquid formulations for oral administration may include suspensions, solutions for internal use, emulsions and syrups, and may contain various excipients such as wetting agents, sweeteners, aromatics and preservatives in addition to generally used simple diluents such as water and liquid paraffin, but may not be limited thereto.

In an embodiment of the present disclosure, formulations for parenteral administration may include sterilized aqueous solutions, water-insoluble excipients, suspensions, emulsions, lyophilized preparations and suppositories, but may not be limited thereto. For example, the water insoluble excipients or suspensions may contain propylene glycol, polyethylene glycol, vegetable oil such as olive oil, injectable ester such as ethylolate, and the like, but may not be limited thereto. For example, the suppositories may contain witepsol, macrogol, tween 61, cacao butter, laurin butter, glycerol, gelatin, and the like, but may not be limited thereto.

The pharmaceutical composition according to an embodiment of the present disclosure may be a drug composition or a quasi-drug composition.

Through the whole document, the term “quasi-drug” refers to an article having a milder action than drugs, among articles being used for the purpose of diagnosis, treatment, improvement, alleviation, handling or prevention of human or animal diseases. For example, according to Pharmaceutical Affairs Law, the quasi-drugs are those, excluding articles used as drugs, including articles used for the purpose of treating or preventing human or animal diseases and articles which have a mild action on or have no direct influence on the human body.

The quasi-drug composition of the present disclosure may be manufactured in a formulation selected from the group consisting of body cleanser, sanitizer, detergent, kitchen cleanser, detergent for cleaning, toothpaste, mouthwash, wet wipe, cleanser, soap, hand soap, hair cleanser, hair softener, humidifying filler, mask, ointment or filter filler, but may not be limited thereto.

In an embodiment of the present disclosure, the pharmaceutical composition may be administered in a pharmaceutically effective amount. Through the whole document, the term “pharmaceutically effective amount” refers to an amount sufficient to treat or prevent diseases at a reasonable benefit/risk ratio applicable to any medical treatment or prevention. An effective dosage level may be determined depending on factors including severity of the disease, drug activity, a patient's age, body weight, health conditions, gender, sensitivity to the drug, administration time, administration route, and excretion rate of the composition of the present disclosure, duration of treatment, drugs blended with or co-administered with the composition of the present disclosure, and other factors known in the medical field. The pharmaceutical composition of the present disclosure may be administered individually or in combination with a known ingredient for treating intestinal diseases. It is important to administer an amount to obtain a maximum effect in a minimum amount without side effects by considering all of the above-described factors.

In an embodiment of the present disclosure, an administration dose of the pharmaceutical composition may be determined by a person with ordinary skill in the art in view of purpose of use, severity of the disease, a patient's age, body weight, gender, medical history or the kind of a material used as an active ingredient. For example, the pharmaceutical composition of the present disclosure may be administered at a dose of from about 0.1 ng/kg to about 1,000 mg/kg, and preferably, from about 1 ng/kg to about 100 mg/kg per adult, and the administration frequency of the composition of the present disclosure is not particularly limited, but the composition may be administered once a day or several times a day in divided doses. The administration dose or the administration frequency does not limit the scope of the present disclosure in any aspect.

The pharmaceutical composition of the present disclosure may be administered via, but not particularly limited to, intraperitoneal administration, intravenous administration, intramuscular administration, subcutaneous administration, intradermal administration, transdermal patch administration, oral administration, intranasal administration, intrapulmonary administration, rectal administration, etc. depending on the purpose. However, when the pharmaceutical composition is administered via oral administration, it can be administered in an unformulated form, and since the strain Lactobacillus gasseri LM1065 can be denatured or degraded by gastric acid, the composition of oral administration may be coated with an active drug, formulated to be protected from degradation in the stomach, or formulated in the form or an oral patch. Also, the composition may be administered by any device capable of delivering an active ingredient to a target cell.

Hereinafter, the present disclosure will be explained in more detail with reference to Examples. However, the following Examples are illustrative only for better understanding of the present disclosure but do not limit the present disclosure.

### Example 1. NO Assay after LPS Treatment

To identify the capacity to produce NO, which is an important biomarker in immune responses, the following test was conducted.

RAW 264.7 cells, mouse macrophages, were inoculated into a 96-well plate at a concentration of 1×105 cells/well. The culture plate was cultured for 24 hours in an environment of 37° C. and 5% CO2.

Then, the supernatant of the culture plate was removed, followed by treatment with Lactobacillus gasseri at a concentration of 1E+8 CFU/ml and culture in an incubator for 2 hours.

Thereafter, treatment with LPS at a concentration of 1 ug/ml was performed, followed by additional culture for 24 hours.

Finally, 100 ul of the supernatant of the culture plate was seeded to a new culture plate, and, then, the absorbance at 540 nm was measured with an ELISA reader after reaction with a Griess reagent.

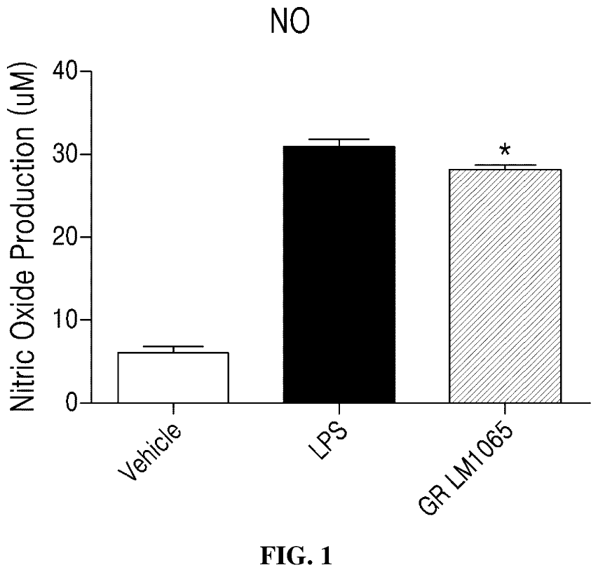

As a result, it was confirmed that, compared to a positive control group (PC) treated only with LPS, additional culture with Lactobacillus gasseri LM1065 induced a statistically significant decrease in production amount of NO at a concentration of 1E+8 CFU/ml (see FIG. 1).

Therefore, the strain of Lactobacillus gasseri LM1065 of the present disclosure can inhibit the production of NO and thus contribute to relieving menstrual pain.

### Example 2: Anti-Inflammatory Gene Expression Capacity Measurement Test Using Real-Time PCR

To identify the expression level of IL-6, which is an anti-inflammatory gene, the following test was conducted.

RAW 264.7 cells, mouse macrophages, were inoculated into a 96-well plate at a concentration of 1×105 cells/well. The culture plate was cultured for 24 hours in an environment of 37° C. and 5% CO2.

Then, the supernatant of the culture plate was removed, followed by treatment with Lactobacillus gasseri at a concentration of 1E+8 CFU/ml and culture in an incubator for 2 hours.

Thereafter, treatment with LPS at a concentration of 1 ug/ml was performed, followed by additional culture for 24 hours.

Then, RNA was extracted using an RNA extraction kit (TaKaRa), and cDNA was synthesized using a high-capacity cDNA reverse transcription kit (Applied Biosystems) and diluted to a final concentration of 5ng/ul.

Finally, real-time PCR was performed using a SYBR Green PCR Master Mix (Applied Biosystems) to compare and identify the expression level of IL-6 gene.

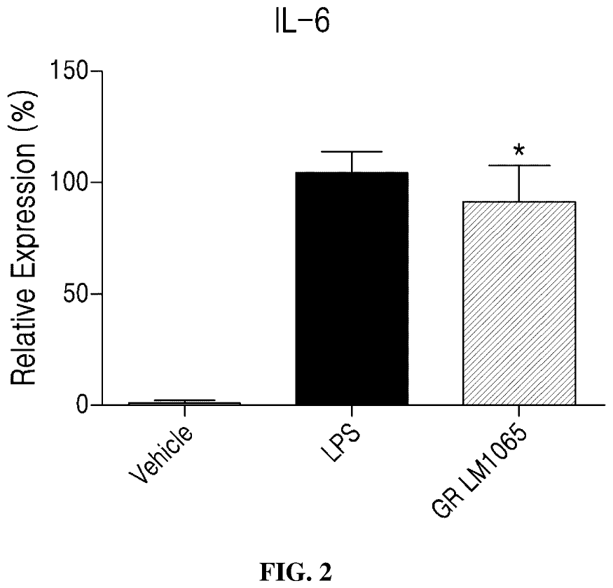

As a result, it was confirmed that, compared to a positive control group (PC) treated only with LPS, additional culture with Lactobacillus gasseri LM1065 induced a statistically significant decrease in expression level of IL-6 gene at a concentration of 1E+8 CFU/ml (see FIG. 2).

Therefore, the strain of Lactobacillus gasseri LM1065 of the present disclosure can inhibit the expression of IL-6 gene and thus contribute to relieving menstrual pain.

### Example 3: PMS Improvement Effect Evaluation Test Using SD Rat

To evaluate an improvement effect appearing when the strain of the present disclosure was repeatedly administered daily for 4 weeks to premenstrual syndrome (PMS)-induced Sprague Dawley (SD) rat models, the following test was conducted.

The rats used in the present example are specific pathogen-free (SPF) rats produced and supplied by CoreTech. The test was conducted on 63 6-week-old female rats in a laboratory set at a temperature of 23±3° C., a relative humidity of 55±15%, a ventilation frequency of 10 to 20 times/hr, a lighting time of 1 hour (turned on at 8:00 a.m. —on during p.m. — off at 8:00 p.m.) and an illumination intensity of 150 to 300 Lux.

A test material was prepared to a predetermined concentration by weighing an appropriate amount of the test material and diluting the test material in sterile water for injection. The test material was administered via oral administration once/day for 4 weeks, and the dosage was calculated to be 10 mL/kg based on the body weight measured on the latest body weight measurement day.

A vaginal cornification (smear cytology) was performed at week 4 after administration of the test material. Blood was collected once/day for 3 days (Day 26, Day 27 and Day 28) from 3 days before the last administration of the test material. Blood collection was performed through jugular vein, and the collected blood was injected into a vacutainer tub containing a clot activator, left at room temperature for about 15 minutes to coagulate, and then centrifuged at 3,000 rpm for 10 minutes for serum isolation. The serum was stored in a deep freezer set at −70° C. or lower until analysis and used for ELISA. At each blood collection time point, the concentrations of PGE1 and PGE2 and the concentration of PGE1/PGE2 were analyzed using a portion of the serum obtained by centrifugation with an ELISA kit. The analysis was performed using a commercially available ELISA kit.

The concentration of DGLA in blood was analyzed by the LC-MS/MS method using the serum collected on the last day of administration of the test material.

The results of the present example were analyzed by using parametric multiple comparison procedures or non-parametric multiple comparison procedures under the assumption of the normality of data. When the result of parametric One-way ANOVA was significant, a post-hoc test was performed through Dunnett's multiple comparison test, and when the result of non-parametric Kruskal-Wallis' H-test was significant, a post-hoc test was performed through Mann-Whitney test. Statistical analysis was performed by using Prism 7.04 (GraphPad Software INC., San Diego, CA, USA), and a p-value of less than 0.05 was determined to be statistically significant.

According to the result of vaginal cornification, no difference in the pattern of keratinocyte changes was observed in estrus among all test groups.

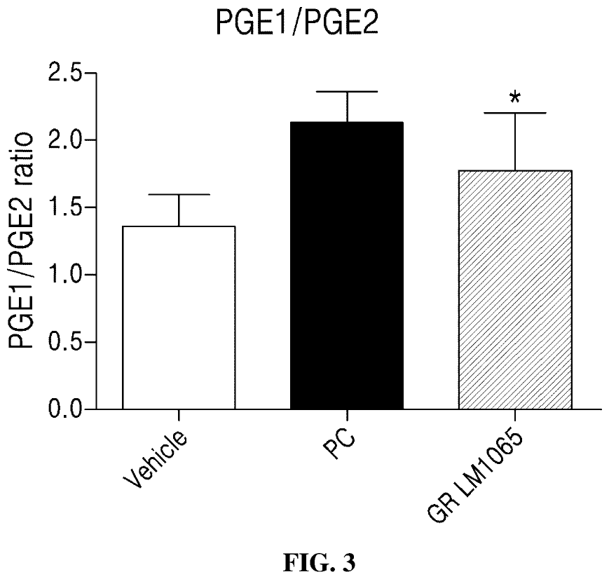

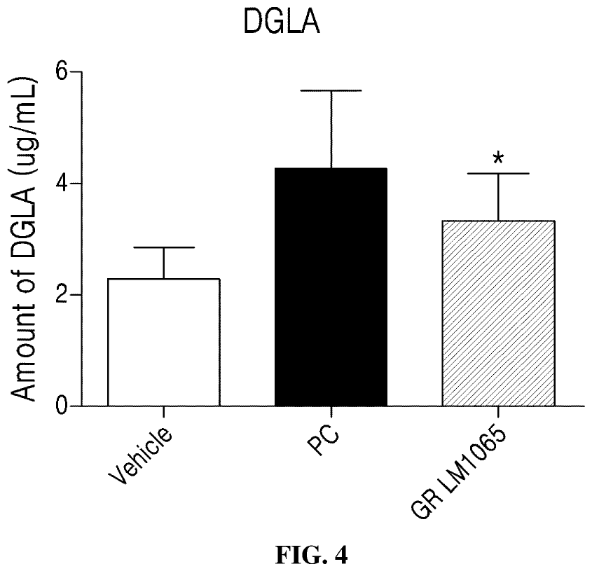

According to the result of ELISA, it was found that the prostaglandin E1/prostaglandin E2 ratio of a Lactobacillus gasseri LM1065-administered group was significantly higher than that of a normal control group (Vehicle) on day 28 (p<0.05, see FIG. 3). According to the result of DGLA analysis, it was found that the DGLA level was significantly higher than that of the normal control group (Vehicle) (p<0.05, see FIG. 4).

When the test material was repeatedly administered to the PMS-induced SD rat models for 4 weeks under the present test conditions, an increase in the prostaglandin E1/prostaglandin E2 ratio and an increase in the concentration of DGLA in blood were observed in the Lactobacillus gasseri LM1065-administered group. It seems that the administration of the test material induces an increase in PGE1 and a decrease in PGE2 to induce the improvement of symptoms in the PMS models. Therefore, the strain of the present disclosure and a composition containing the strain or its culture fluid are effective in improving PMS.

As described above, it was confirmed that the strain of Lactobacillus gasseri LM1065 of the present disclosure can increase the PGE1/PGE2 ratio and promote the production of DGLA, which is an inflammation-reducing substance, and thus contribute to relieving menstrual pain.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A composition comprising a strain of Limosilactobacillus gasseri LM1065 (KCCM13018P).

2. The composition of claim 1,
wherein the strain reduces the production amount of NO in blood.

3. The composition of claim 1,
wherein the strain reduces TL-6 gene expression.

4. The composition of claim 1,
wherein the strain increases the ratio of prostaglandin E1/prostaglandin E2 (PGE1/PGE2) in blood.

5. The composition of claim 1,
wherein the strain increases the level of DGLA in blood.

6. A food composition of relieving menstrual pain, comprising:
one or more of a strain of Lactobacillus gasseri LM1065 (KCCM13018P), and culture fluid, fragments and extracts of the strain as active ingredients.

7. A pharmaceutical composition of preventing or treating premenstrual syndrome, comprising:
one or more of a strain of Lactobacillus gasseri LM1065 (KCCM13018P), and culture fluid, fragments and extracts of the strain as active ingredients.

8. The pharmaceutical composition of preventing or treating premenstrual syndrome of claim 7,
wherein the premenstrual syndrome includes one or more selected from the group consisting of edema, breast pain, digestive disorders, headache, back pain, lower abdominal pain, abdominal distention, constipation, diarrhea, depression, and insomnia.
